(19) United States
(12) Patent Application Publication
(10) Pub. No.: US 2026/0174388 A1
(43) Pub. Date: Jun. 25, 2026

(54) Device for Detecting and Illuminating the Vasculature Using an FPGA

(71) Applicant: AccuVein Inc., Medford, NY (US)

(72) Inventors: Joseph Zott, Menlo Park, CA (US); Fred Wood, Medford, NY (US); Dmitriy Yavid, Stony Brook, NY (US); Seung P. Kim, San Francisco, CA (US); Klaus Zietlow, Piedmont, CA (US)

(73) Assignee: AccuVein Inc., Medford, NY (US)

(21) Appl. No.: 19/439,637

(22) Filed: Jan. 5, 2026

Related U.S. Application Data
(63) Continuation of application No. 17/991,924, filed on Nov. 22, 2022, now Pat. No. 12,558,030.
Application No. 17/991,924 is a continuation of application No. 16/789,459, filed on Feb. 13, 2020, now Pat. No. 11,510,617.

(51) Int. Cl. A61B 5/00; A61B 90/00; A61M 5/42; G02B 26/10

(52) U.S. Cl. CPC A61B 5/489; A61B 5/0033; A61B 5/0062; A61B 5/0082; A61B 5/743; A61B 2090/366; A61M 5/427; G02B 26/101

(57) ABSTRACT

An imager obtains and projects a vein image onto a patient and includes: an infrared laser; visible light laser; x-mirror; x-mirror driver; y-mirror; y-mirror driver; field programmable gate array (FPGA); photodiode; and front end circuit. The FPGA controls the lasers, x-mirror driver, and y-mirror driver to control the x-mirror and y-mirror to scan the light, and receives feedback of a state of the lasers, forming a closed control loop. The photodiode converts the reflected infrared vein image into an analog signal, which is processed by the FPGA including: performing an X-direction sum of pixel data for a plurality of boundary windows and placing it into an X-sum buffer, performing a Y-direction sum of pixel data and placing it into a Y-sum buffer, and using a total sum generator to obtain box filter output for each of the plurality of windows, being the X-sum buffer added to the Y-sum buffer.

### CROSS-REFERENCE TO RELATED APPLICATIONS

This application is a continuation of U.S. patent application Ser. No. 17/991,924, filed on Nov. 22, 2022, which is a continuation of U.S. patent application Ser. No. 16/789,459, filed on Feb. 13, 2020, which issued as U.S. Pat. No. 11,510,617 on Nov. 29, 2022, which is a continuation of U.S. patent application Ser. No. 15/679,277, filed on Aug. 17, 2017, which issued as U.S. Pat. No. 10,568,518 on Feb. 5, 2020, which is a continuation of U.S. patent application Ser. No. 14/723,674, filed on May 28, 2015, which issued as U.S. Pat. No. 9,782,079 on Oct. 10, 2017, which is a continuation of U.S. patent application Ser. No. 13/957,767, filed on Aug. 2, 2013, which issued as U.S. Pat. No. 9,072,426 on Jul. 7, 2015, which claims priority on U.S. Provisional Patent Application Ser. No. 61/678,726, filed on Aug. 2, 2012, with the disclosures of each being incorporated herein by reference.

### BACKGROUND

### Summary

A laser based vascular illumination system utilizing a FPGA for detecting vascular positions, processing an image of such vasculature positions, and projecting the image thereof onto the body of a patient.

### BRIEF DESCRIPTION

FIG. 1 Block diagram of a system for detecting and illuminating the vasculature in a patient.

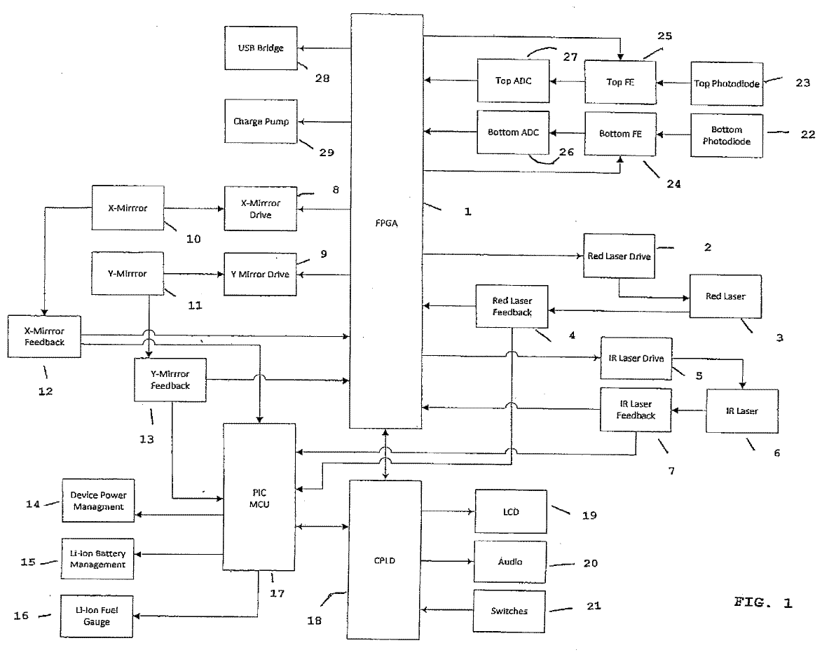

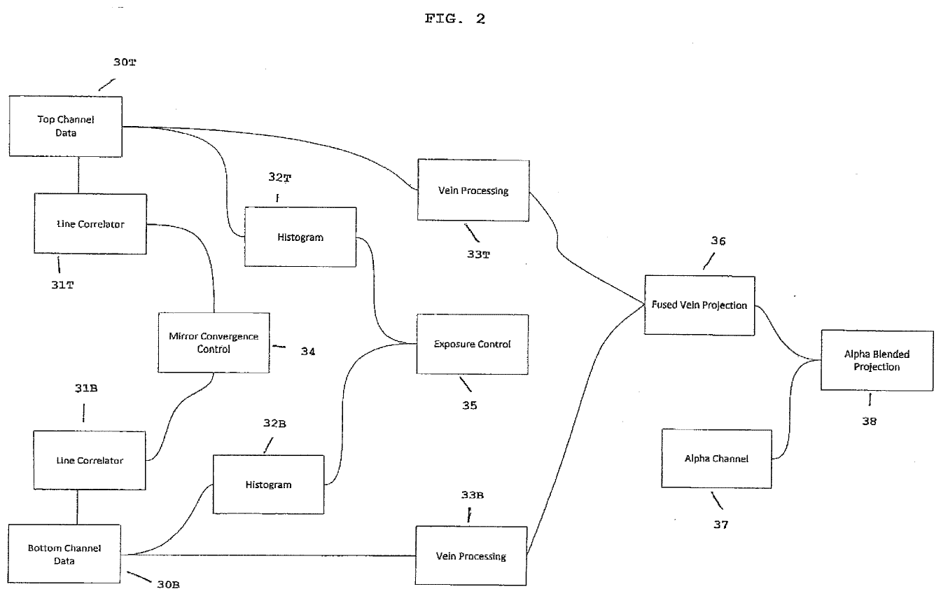

FIG. 2 Shows the signal processing flow of the FPGA.

FIG. 3 shows the internal bus architecture of the FPGA.

FIG. 4 shows details of the vein processing.

FIG. 5 shows the vein processing at the boundary of the image frames.

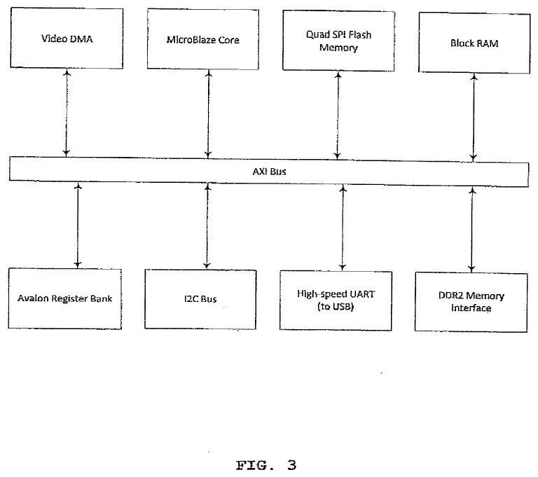

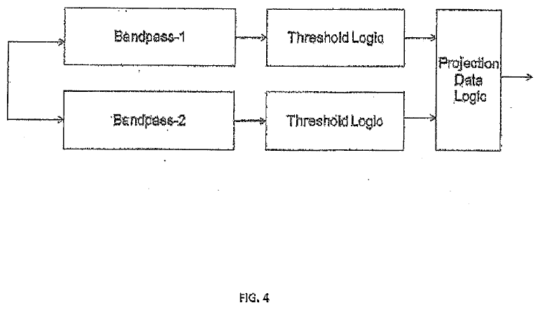

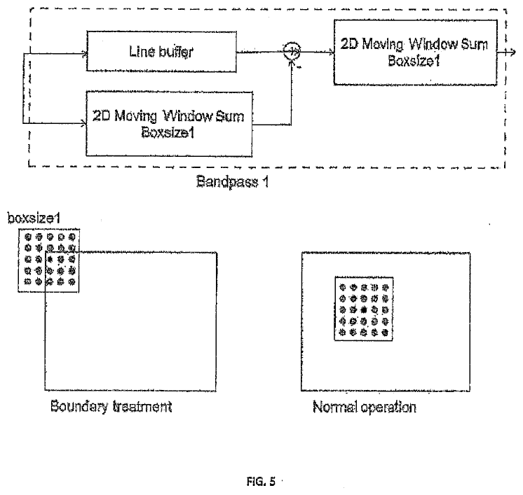

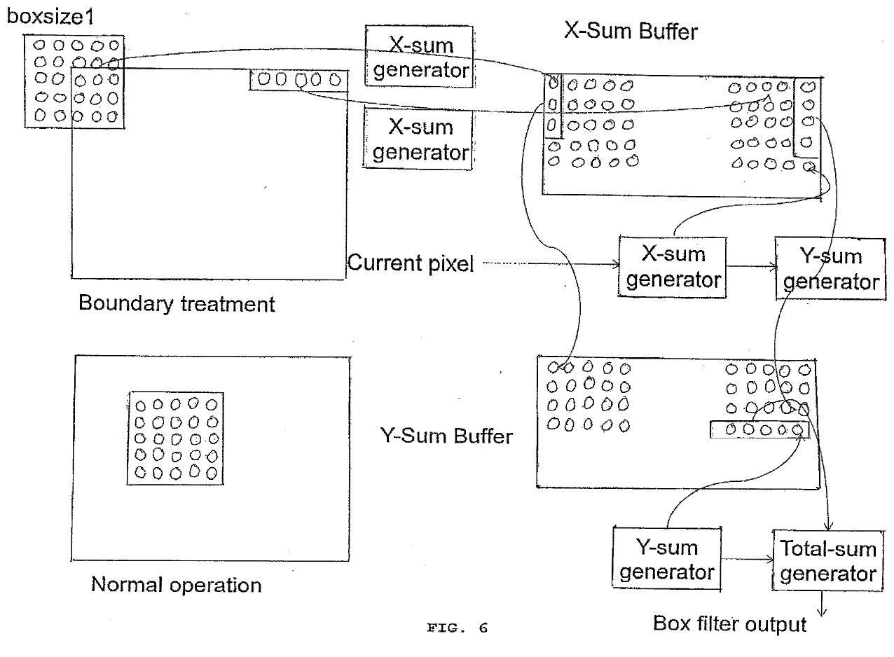

FIG. 6 shows further detail of the vein processing at the boundary of the image frames.

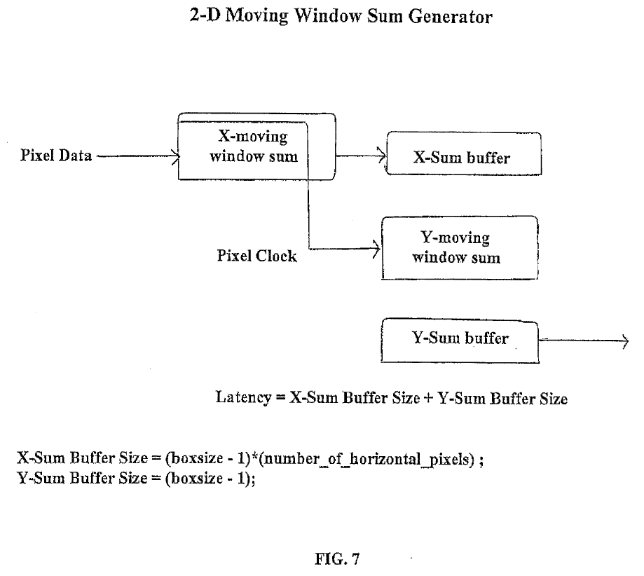

FIG. 7 2-D Moving Window Sum Generator.

FIG. 8 shows a X-sum generator.

### DETAILED DESCRIPTION

FIG. 1 shows a block diagram of a system for detecting and illuminating the vasculature in a patient.

The system shown in the block diagram of FIG. 1 is used for detecting the location of veins on a patient and illuminating the veins.

The disclosures of U.S. patent application Ser. No. 12/804,506, now issued as U.S. Pat. No. 8,463,364 are incorporated herein by reference.

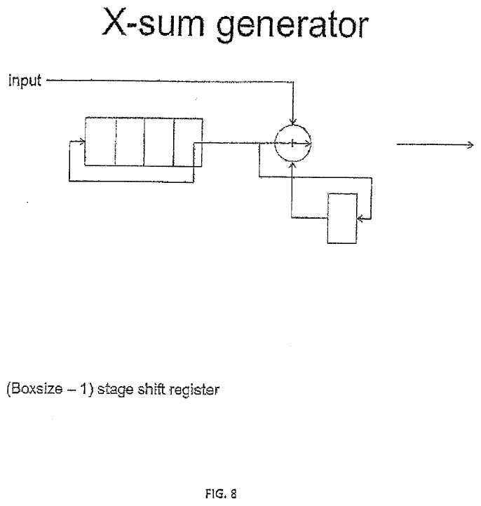

In a preferred embodiment, FIGS. 30-47 of application Ser. No. 12/804,506 illustrates an assembly of a housing that may be used in the present invention. In the present invention, circuit boards 43, 44 and 15 of application Ser. No. 12/804,506 may be modified to contain the circuitry described by the block diagram in FIG. 1. The remainder of the device in FIGS. 30-47 can remain substantially the same.

In FIG. 1 an FPGA 1 (field programmable gate array) is configured to control a red laser drive 2 which in turn drives a red laser 3. The output of the red laser 3 is controlled in a manner so as to illuminate the detected veins. A red laser feedback 4 detects the output of the red laser 3 and sends the information to the FPGA 1. Accordingly, a closed loop is formed whereby the FPGA 1 can both drive the Red laser 3 and receive feedback as to the red laser 3 state.

In FIG. 1 an FPGA 1 (field programmable gate array) is configured to control a red laser drive 2 which in turn drives a red laser 3. The output of the red laser 3 is controlled in a manner so as to illuminate the detected veins. A red laser feedback 4 detects the output of the red laser 3 and sends the information to the FPGA 1. Accordingly, a closed loop is formed whereby the FPGA 1 can both drive the Red laser 3 and receive feedback as to the red laser 3 state.

FPGA 1 outputs data to an IR laser drive 5 which in turn drives an IR laser 6. The output of the IR laser 6 is controlled to output an intensity of IR light, aimed at the area of the body where veins are located, sufficient to detect the veins. An IR laser feedback 7 detects the output of the IR laser 6 and sends the information to the FPGA 1. Accordingly, a closed loop is formed whereby the FPGA 1 can both drive the IR Laser 6 and receive feedback as to the IR laser 6 state.

FPGA 1 communicates to both a x-mirror drive 8 and a y-mirror drive 9 to drive x-mirror 10 and y-mirror 11 in such a manner that a raster pattern is formed on the patient when the Red laser 3 and the IR laser 6 are coaxially projected thereon. X-mirror feedback 12 and y-mirror feedback 13 detect the positions of the x-mirror 10 and y-mirror 11, respectively, and communicates such information to the FPGA1.

Top photodiode 23 and bottom photodiode 22 receive the IR Laser 6 reflected off the patient, converts the light into an analog signal which is provided to Top FE 25 and Bottom FE 24, and then to Top ADC 27 and bottom ADC 25, respectively. The top FE 25 and the bottom FE 24 are front end circuits that provide analog filtering, gain control and threshold of the analog signals. The Top ADC 27 and bottom ADC 26 are analog to digital converters that convert the analog signals to digital representations thereof to be communicated to the FPGA 1. Control lines are provided from the FPGA 1 to the top FE 25 and the bottom FE 24 to set parameters such as, for example, gain control and analog filtering.

From a mechanical standpoint, the red laser 3 and the IR laser 6 are co axially aligned and projected off of mirrors X-mirror 10 and Y-mirror 11 to form a pattern, such as for example, a raster pattern on the patient. The IR laser 6 reflects off the patient and is received by top photodiode 23 and photodiode 22. The reflected IR light contains information as to the location of the veins (IR light is absorbed by the blood in the veins and therefore the amount or reflected IR light is lower when the IR laser 6 is aimed at a vein. The FPGA 1 time sequentially receives in the signal form the top ADC 27 and the bottom ADC and can form two partial and/or full frame images of the reflected IR light (hereinafter a top channel data and a bottom channel data wherein the top channel data is received from the top ADC 27 and the bottom channel data is received from the bottom ADC). The FPGA 1 processes one or both of the partial and/or full image to detect and enhance the image of the veins. The enhanced image is time sequentially projected by the Red laser 3 onto the patient.

A CPLD is provided for controlling an LCD 19 with displays user information related to the operating status of the device. It also controls an audio 20 output to provide audible tones to the user. Finally the CPLD 18 controls the switches 21 on the unit for turning on and off the units as well as selecting user modes and entering data.

A microprocessor PIC MCU 17 is provided for receiving and monitoring the IR laser feedback 7 signal, the red laser feedback 4 signal, the x-mirror feedback 12 signal and the y-mirror feedback 13 signal. Since these signals are also provided to the FPGA 1, redundancy monitoring of the signals is provided by the PIC MCU 17. This is particularly important when regulatory requirements require redundant monitoring of the laser power and movement to comply with safety requirements. The NC MCU 17 also monitors the device power management 14, the Li-ion Battery management 15 circuitry and the Li-ion Fuel gauge 16.

FIG. 2 Shows an example of the signal processing flow of the FPGA.

FIG. 2 shows an embodiment of the signal processing algorithm of the FPGA of FIG. 1. As described with reference to FIG. 1, the image of the reflected IR laser 6 is time sequentially stored in the FPGA 1 as top channel data 30T and bottom channel data 30B.

The X-mirror 10 oscillates about a single axis to move the laser beam from the IR laser 6 to form a line. The beam moves first in one direction and then back in the other direction. It is critical that the left to right image data be in convergence with the right to left data. The top line correlator 31T measures the shift in the convergence of the top channel data 30T and supplies the information to the mirror convergence control 34. Similarly, the bottom line correlator 31B measures the shift in the convergence of the bottom channel data 30B and supplies the information to the mirror convergence control 34. The mirror convergence control 34 can adjust the control signals provided from the FPGA 1 to the x-mirror drive 8 so as to converge the data.

A top histogram 32T receives the top channel data 30T and generates a histogram based upon an entire frame of the top channel data 30T. Similarly, a bottom histogram 32B receives the top channel data 30B and generates a histogram based upon an entire frame of the bottom channel data 30B. The histograms contain information describing the characteristics of the images, including but not limited to contrast and intensity levels. The top histogram 32T and the bottom histogram 32B are provided to exposure control 35. Exposure control 35 communicates appropriate signals the IR laser drive 5 to adjust the power of the IR laser 6 on a frame by frame basis until the histograms indicate appropriate images. The exposure control 35 also communicates with the top FE 25 and bottom FE 24 to adjust parameters such as setting thresholds and setting electrical gain.

A top vein processing 33T block receives the top channel data 30T and performs image processing to detect vein patterns and provides the enhanced vein image to fused vein projection 36. Similarly, bottom vein processing 33B block receives the bottom channel data 30B and performs image processing to detect vein patterns and provides the enhanced vein image to fused vein projection 36. The fused vein projection 36 forms a single image and communicates the Image to the alpha blended projection 38. The fused vein projection 36 can form the single image by merging the images from the top vein processing 33T and bottom vein processing 33B. Alternative, the fused vein projection 36 can simply select the best image received from the top vein processing 33T and the bottom vein processing 33B.

Alpha channel 37 forms an image that contains graphical data, such as text or characters. Alpha channel 37 and fused vein projection 36 are provided to alpha blended projection 38 with drives the IR laser drive 5 to display an image which is the combination of the fused vein projection 36 and the alpha channel 37.

FIG. 3 shows an example of the internal bus architecture of the FPGA.

FIG. 4 shows details of the top vein processing 33T and bottom vein processing 33B.

FIG. 5 shows the vein processing at the boundary of the image frames.

FIG. 6 shows further detail of the vein processing at the boundary of the image frames.

FIG. 7 shows the 2-D Moving Window Sum Generator.

FIG. 8 shows a X-sum generator.

1. A vein imaging device configured to obtain and project an image of subcutaneous veins onto a patient to overlie the imaged veins, said vein imaging device comprising:
a first laser configured to emit a beam of light at an infrared wavelength;
a second laser configured to emit a beam of light at a visible wavelength;
an x-mirror;
an x-mirror driver;
a y-mirror;
a y-mirror driver;
a field programmable gate array (FPGA), said FPGA configured to control said x-mirror driver and said y-mirror driver to control said first laser, said second laser, said x-mirror and said y-mirror to selectively scan each of said beam of light at said infrared wavelength and said beam of light at said visible wavelength onto the patient in a first direction and then in a second direction, to form lines, and to scan said lines in a third direction to thereby form a pattern of said infrared wavelength of light and said visible wavelength of light;
wherein said FPGA is further configured to receive feedback of a state of said first laser and a state of said second laser, to form a closed loop to control an output of said first laser and to control an output of said second laser;
a photodiode configured to receive a vein image formed from said scanned pattern of said infrared wavelength of light, and further configured to convert the received vein image into an analog signal;
a front end circuit, said front end circuit configured to provide analog filtering, gain control, and threshold of the analog signal;
an analog to digital converter (ADC), said ADC configured to convert the analog signal to a digital signal;
wherein said FPGA is further configured to receive the digital signal and to perform vein image processing of an image frame of said received vein image, said vein image processing comprising: said FPGA being configured to collect pixel data from said photodiode, to perform an X-direction sum of pixel data for a plurality of boundary windows comprising: a matrix of pixels, and to place said X-direction sum into an X-sum buffer, to perform a Y-direction sum of pixel data for said plurality of boundary windows, and to place said Y-direction sum into a Y-sum buffer, and to use a total sum generator to obtain box filter output for each of said plurality of windows being the X-sum buffer added to the Y-sum buffer;
wherein said FPGA is further configured to control said x-mirror driver and said y-mirror driver to control said first laser, said second laser, said x-mirror and said y-mirror, to scan said visible wavelength within said beam for projection of said processed vein image onto the patient; and
wherein said FPGA is further configured to set said analog filtering and gain, to control said front end circuit.

2. The imaging device according to claim 1, wherein said FPGA is further configured to provide boundary treatment image processing, wherein a portion of one or more of said plurality of boundary windows extend beyond said image frame.

3. The imaging device according to claim 2, further comprising a pixel clock configured to time sequentially store said image in said FPGA.

4. The imaging device according to claim 1, further comprising: a pixel clock.
